(19) United States
(12) Patent Application Publication
(10) Pub. No.: US 2016/0348051 A1
(43) Pub. Date: Dec. 1, 2016

(54) CULTURE CONTAINER, GEL MATERIAL, AND CULTURE SYSTEM

(71) Applicant: Seiko Epson Corporation, Tokyo (JP)

(72) Inventors: Satomi YOSHIOKA, Shiojiri (JP); Hiroshi YAGI, Tatsuno (JP)

(21) Appl. No.: 15/145,202

(22) Filed: May 3, 2016

(30) Foreign Application Priority Data
May 27, 2015 (JP) 2015-107155

(51) Int. Cl. C12M 1/12

(52) U.S. Cl. CPC C12M 25/14

(57) ABSTRACT

A culture container includes a gel section constituted by a material containing a stimulus-responsive gel which expands or contracts in response to a stimulus by a given substance in at least a portion of a part coming into contact with a culture solution.

### BACKGROUND

1. Technical Field

The present invention relates to a culture container, a gel material, and a culture system.

2. Related Art

In the production of pharmaceutical products, and in the fields of gene therapy, regenerative medicine, immunotherapy, etc., it has been demanded that cells, tissues, microorganisms, etc. should be efficiently cultured in a large amount in an artificial environment.

In order to perform efficient culture, monitoring of the concentration or the like of a metabolite resulted from the culture in a culture container is required.

As a device that performs monitoring of a metabolite resulted from the culture, for example, there has been known a device that performs monitoring of lactic acid as a metabolite (see, for example, Japanese Patent No. 3078966). In such a device, an enzyme electrode is used for detecting the metabolite. However, an electrode method using an enzyme has problems as described below.

For example, the price of an enzyme which is a biological reagent is high, and depending on the storage environment such as temperature and humidity, the enzyme molecule is degraded, which may affect the quantitativeness. Further, the enzyme varies in activity among production lots or manufacturers or depending on the storage environment or the like, and therefore, it is necessary to perform calibration using standard solutions having known concentrations before using the enzyme electrode. In addition, a reaction with oxygen during an enzymatic reaction is essential for an enzyme used for an enzyme electrode such as lactate oxidase, and therefore, in a state where oxygen is lacking (in an anaerobic environment, particularly when glycolytic metabolism is enhanced, the oxygen demand is increased due to excessive cell proliferation), an error in the measurement using an enzyme electrode is likely to be large. Further, in the case where an enzyme electrode is used, it is necessary to draw out a wire to the outside of the culture container due to the placement of the electrode in the culture solution, however, contamination with bacteria or biological toxins from the outside in the culture should be avoided, and therefore, it is necessary to strictly maintain the sealing of the circumference of the wire, and therefore, the handleability of the culture container is lowered, and the cost is significantly increased.

### SUMMARY

An advantage of some aspects of the invention is to provide a culture container capable of favorably performing monitoring of the inside while preventing contamination of the inside, to provide a gel material capable of being favorably applied to a culture container capable of favorably performing monitoring of the inside while preventing contamination of the inside, and also to provide a culture system capable of favorably performing monitoring of the inside of a culture container while preventing contamination of the inside of the culture container.

A culture container according to an aspect of the invention includes a gel section constituted by a material containing a stimulus-responsive gel which expands or contracts in response to a stimulus by a given substance in at least a portion of a part coming into contact with a culture solution.

According to this configuration, a culture container capable of favorably performing monitoring of the inside while preventing contamination of the inside can be provided.

In the culture container according to the aspect of the invention, it is preferred that the culture container includes a container main body and the gel section, and the gel section is provided on an inner bottom surface of the container main body.

In the culture container according to the aspect of the invention, it is preferred that the culture container includes a container main body and the gel section, and at least the part of the container main body where the gel section is provided is constituted by a material having light transmittance.

In the culture container according to the aspect of the invention, it is preferred that the stimulus-responsive gel includes a first polymer having an OH group and a second polymer having a phenylboronic acid structure as polymer materials.

In the culture container according to the aspect of the invention, it is preferred that the stimulus-responsive gel is transformed to a second state where the bond between the OH group possessed by the first polymer and the phenylboronic acid structure possessed by the second polymer is dissociated by reacting the phenylboronic acid structure possessed by the second polymer with lactic acid.

In the culture container according to the aspect of the invention, it is preferred that the first polymer contains N-hydroxyethylacrylamide as a constituent component.

In the culture container according to the aspect of the invention, it is preferred that the second polymer contains a monomer having a phenylboronic acid structure as a constituent component.

In the culture container according to the aspect of the invention, it is preferred that the second polymer contains 3-acrylamidophenylboronic acid as a constituent component.

In the culture container according to the aspect of the invention, it is preferred that when the content of the first polymer is represented by X₁ (mass %) and the content of the second polymer is represented by X₂ (mass %), the following relationship is satisfied: 0.2≦X₂/X₁≦8.

In the culture container according to the aspect of the invention, it is preferred that the gel section contains the stimulus-responsive gel and fine particles having an average particle diameter of 10 nm or more and 1000 nm or less.

A gel material according to an aspect of the invention is a material constituting a region containing at least a portion of a part coming into contact with a culture solution of a culture container, and includes a stimulus-responsive gel which expands or contracts in response to a stimulus by a given substance.

According to this configuration, a gel material capable of being favorably applied to a culture container capable of favorably performing monitoring of the inside while preventing contamination of the inside can be provided.

A culture system according to an aspect of the invention includes the culture container according to the aspect of the invention, and a detection unit that detects the state of the gel section.

According to this configuration, a culture system capable of favorably performing monitoring of the inside of a culture container while preventing contamination of the inside of the culture container can be provided.

### BRIEF DESCRIPTION OF THE DRAWINGS

The invention will be described with reference to the accompanying drawings, wherein like numbers reference like elements.

FIG. 1 is a schematic perspective view for illustrating a preferred embodiment of a culture container.

FIG. 2 is a schematic perspective view for illustrating a preferred embodiment of a culture system.

### DESCRIPTION OF EXEMPLARY EMBODIMENTS

Hereinafter, preferred embodiments will be described in detail with reference to the accompanying drawings.

### Culture Container

Hereinafter, a culture container will be described.

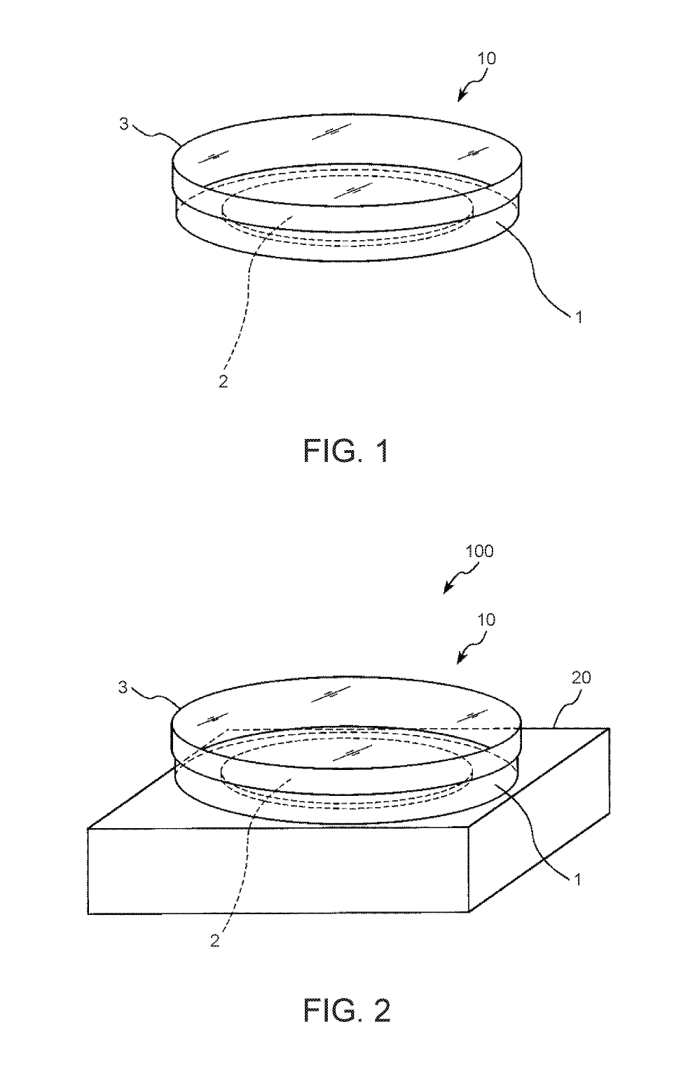

FIG. 1 is a schematic perspective view for illustrating a preferred embodiment of a culture container.

As shown in FIG. 1, a culture container 10 includes a container main body 1 and a gel section 2 constituted by a material containing a stimulus-responsive gel which expands or contracts in response to a stimulus by a given substance (specific component).

The gel section 2 is provided in at least a portion of a part coming into contact with a culture solution.

According to such a configuration, monitoring of a given substance contained in the culture solution (for example, a component contained in the initial culture solution, a metabolite resulted from the culture, etc.) can be favorably performed. In particular, it is not necessary to insert or pull out an enzyme electrode or the like, and monitoring can be performed in a closed system, and therefore, monitoring of a given substance can be favorably performed while preventing contamination of the inside of the culture container 10. Further, the state of the cultured cells can be recognized instantaneously, and therefore, it is possible to appropriately adjust the culture conditions, and thus, the culture efficiency can be made excellent.

The container main body 1 has a function to house a culture solution, cultured cells, and the like.

The container main body 1 may be constituted by any material, however, it is preferred that at least a part where the gel section 2 is provided is constituted by a material having light transmittance.

According to this, the state of the inside of the culture container 10 can be favorably observed from the outside.

Examples of the material having light transmittance include various types of glass materials and various types of plastic materials.

The shape of the container main body 1 is not particularly limited, however, in the configuration shown in the drawing, the container main body 1 has a dish shape (a petri dish shape). According to this, the culture efficiency can be made excellent, and also the state of the gel section 2 can be favorably observed from the outside.

The stimulus-responsive gel constituting the gel section 2 expands or contracts in response to a stimulus by a given substance (specific component).

The given stimulus to which the stimulus-responsive gel responds varies depending on the constituent material or the like of the stimulus-responsive gel, however, examples thereof include proteins, sugars, uric acid, urea, lactic acid, various types of hormones, various types of ionic substances, and various types of metals.

In particular, it is known that when anaerobic metabolism is enhanced in the culture solution, the concentration of lactic acid in the culture solution is increased. Therefore, by ascertaining the concentration of lactic acid as the given substance based on the change in the state of the stimulus-responsive gel, the state of the cultured cells can be recognized instantaneously while maintaining the sterile conditions in a non-contact manner with the culture solution. As a result, it is possible to appropriately adjust the culture conditions, and thus, the culture efficiency can be made more excellent.

The gel section 2 may be provided in at least a portion of a part coming into contact with the culture solution, however, in the configuration shown in the drawing, the gel section 2 is provided on an inner bottom surface of the container main body 1.

According to this, the gel section 2 can be favorably brought into contact with the culture solution, and thus, a stimulus by a given substance can be more stably detected. In addition, the state of the gel section 2 can be favorably observed from the outside.

The shape of the gel section 2 is not particularly limited, but is preferably a sheet shape.

According to this, while suppressing the increase in the size of the culture container 10, a housing space for the culture solution and the like in the culture container 10 can be more effectively ensured.

The volume of the gel section 2 in a contracted state is preferably 0.1 mm³ or more and 3600 mm³ or less, more preferably 0.2 mm³ or more and 900 mm³ or less.

According to this, while reducing the size of the gel section 2, the stimulus detection accuracy can be made more excellent. Further, from the viewpoint of resource saving, the above configuration is preferred.

In the invention, the gel section may be provided, for example, on the entire inner bottom surface, a side face portion, or the like of the container main body. In addition, the gel section can be favorably applied also to a large-size culture container, and therefore, the area of the gel section is not limited to values in the above range.

The thickness of the gel section 2 in a contracted state is not particularly limited, but is preferably 5 μm or more and 5000 μm or less, more preferably 7 μm or more and 1000 μm or less.

According to this, while making the durability and reliability of the gel section 2 and the culture container 10 sufficiently excellent, the thickness and size of the gel section 2 can be reduced. Further, the stimulus detection accuracy can be made more excellent. In addition, the flexibility of the gel section 2 can be made more excellent, and for example, the shape followability to the container main body 1 can be made more excellent, and therefore, the adhesiveness between the container main body 1 and the gel section 2 can be made excellent, and thus, the stimulus detection accuracy can be stably made excellent.

The material (gel material) constituting the gel section 2 will be described in detail later.

The culture container 10 of this embodiment further includes a lid 3.

According to this, placement of the culture solution or the like in the inside of the culture container 10 can be easily performed, and also contamination of the inside of the culture container 10 with a foreign substance can be more effectively prevented.

The lid 3 may be constituted by any material, but is preferably constituted by a material having light transmittance.

According to this, the state of the inside of the culture container 10 can be favorably observed from the outside.

Examples of the material having light transmittance constituting the lid 3 include various types of glass materials and various types of plastic materials.

### Gel Material

Hereinafter, the gel material (the material containing a stimulus-responsive gel) constituting the gel section 2 will be described in detail.

The gel material constituting the gel section 2 contains at least a stimulus-responsive gel which reversibly expands or contracts in response to a stimulus by a given substance.

According to this, monitoring of a given substance contained in the culture solution (for example, a component contained in the initial culture solution, a metabolite resulted from the culture, etc.) can be favorably performed. In particular, it is not necessary to insert or pull out an enzyme electrode or the like, and monitoring can be performed in a closed system, and therefore, monitoring of a given substance can be favorably performed while preventing contamination of the inside of the culture container 10. Further, the state of the cultured cells can be recognized instantaneously, and therefore, it is possible to appropriately adjust the culture conditions, and thus, the culture efficiency can be made excellent.

### Stimulus-Responsive Gel

The stimulus-responsive gel may be constituted by any material as long as it responds to a stimulus by a given substance, but is generally constituted by a material containing a polymer material having a crosslinked structure and a solvent.

### Polymer Material

The polymer material constituting the stimulus-responsive gel is an important component for detecting a specific stimulus, and the structure thereof varies depending on the type of the stimulus to be detected.

The polymer material constituting the stimulus-responsive gel is not particularly limited, and can be selected according to the stimulus to be detected.

Hereinafter, specific examples of the polymer material constituting the stimulus-responsive gel will be described.

As the polymer material constituting the stimulus-responsive gel, for example, a polymer material obtained by reacting a monomer, a polymerization initiator, a crosslinking agent, etc. can be used.

Examples of the monomer include acrylamide, N-methylacrylamide, N-isopropylacrylamide, N,N-dimethylacrylamide, N,N-dimethylaminopropylacrylamide, various quaternary salts of N,N-dimethylaminopropylacrylamide, acryloylmorpholine, various quaternary salts of N,N-dimethylaminoethylacrylate, acrylic acid, various alkyl acrylates, methacrylic acid, various alkyl methacrylates, 2-hydroxyethylmethacrylate, glycerol monomethacrylate, N-vinylpyrrolidone, acrylonitrile, styrene, polyethylene glycol diacrylate, 1,6-hexanediol diacrylate, neopentyl glycol diacrylate, tripropylene glycol diacrylate, polypropylene glycol diacrylate, 2,2-bis[4-(acryloxydiethoxy)phenyl]propane, 2,2-bis[4-(acryloxypolyethoxy)phenyl]propane, 2-hydroxy-1-acryloxy-3-methacryloxypropane, 2,2-bis[4-(acryloxypolypropoxy)phenyl]propane, ethylene glycol dimethacrylate, diethylene glycol dimethacrylate, triethylene glycol dimethacrylate, polyethylene glycol dimethacrylate, 1,3-butylene glycol dimethacrylate, 1,6-hexanediol dimethacrylate, neopentyl glycol dimethacrylate, polypropylene glycol dimethacrylate, 2-hydroxy-1,3-dimethacryloxypropane, 2,2-bis[4-(methacryloxyethoxy)phenyl]propane, 2,2-bis[4-(methacryloxyethoxydiethoxy)phenyl]propane, 2,2-bis[4-(methacryloxyethoxypolyethoxy)phenyl]propane, trimethylolpropane trimethacrylate, tetramethylolmethane trimethacrylate, trimethylolpropane triacrylate, tetramethylolmethane triacrylate, tetramethylolmethane tetraacrylate, dipentaerythritol hexaacrylate, N,N′-methylenebisacrylamide, N,N′-methylenebismethacrylamide, diethylene glycol diallyl ether, and divinylbenzene.

Further, examples of a functional group which can interact with a sugar include a boronic acid group (particularly, a phenylboronic acid group), and therefore, a monomer having a boronic acid group may be used. Examples of such a boronic acid group-containing monomer include acryloylaminobenzeneboronic acid, methacryloylaminobenzeneboronic acid, and 4-vinylbenzeneboronic acid.

In the case where an ionic substance (particularly, an ionic substance containing a calcium ion) is detected as a given substance (specific component), a crown ether group-containing monomer (particularly, a benzocrown ether group-containing monomer) such as 4-acrylamidobenzo-18-crown 6-ether, acryloyl aminobenzocrown ether, methacryloyl aminobenzocrown ether, or 4-vinylbenzocrown ether can be preferably used as the monomer.

In the case where an ionic substance such as sodium chloride is detected as a given substance (specific component), 3-acrylamidophenylboronic acid, vinylphenylboronic acid, acryloyloxyphenylboronic acid, N-isopropylacrylamide (NIPAAm), ethylenebisacrylamide, N-hydroxyethylacrylamide, or the like can be preferably used as the monomer. In particular, in the case where an ionic substance such as sodium chloride is detected as a given substance (specific component), it is preferred to use at least one monomer selected from the group consisting of 3-acrylamidophenylboronic acid, vinylphenylboronic acid, and acryloyloxyphenylboronic acid, and at least one monomer selected from the group consisting of N-isopropylacrylamide (NIPAAm), ethylenebisacrylamide, and N-hydroxyethylacrylamide in combination as the monomer.

In the case where lactic acid is detected as a given substance (specific component), 3-acrylamidophenylboronic acid, vinylphenylboronic acid, acryloyloxyphenylboronic acid, N-isopropylacrylamide (NIPAAm), ethylenebisacrylamide, N-hydroxyethylacrylamide, or the like can be preferably used as the monomer. In particular, in the case where lactic acid is detected as a given substance (specific component), it is preferred to use at least one monomer selected from the group consisting of 3-acrylamidophenylboronic acid, vinylphenylboronic acid, and acryloyloxyphenylboronic acid, and at least one monomer selected from the group consisting of N-isopropylacrylamide (NIPAAm), ethylenebisacrylamide, and N-hydroxyethylacrylamide in combination as the monomer.

The polymerization initiator can be appropriately selected according to, for example, the polymerization method thereof. Specific examples thereof include compounds which generate radicals by ultraviolet light including hydrogen peroxide, persulfates such as potassium persulfate, sodium persulfate, and ammonium persulfate, azo-based initiators such as 2,2′-azobis(2-amidinopropane) dihydrochloride, 2,2′-azobis(N,N′-dimethyleneisobutylamidine) dihydrochloride, 2,2′-azobis{2-methyl-N-[1,1-bis(hydroxymethyl)-2-hydroxyethyl]propionamide}, 2,2′-azobis[2-(2-imidazolin-2-yl)propane]dihydrochloride, 4,4′-azobis(4-cyanovaleric acid), 2,2′-azobisisobutyronitrile, 2,2′-azobis(2,4′-dimethylvaleronitrile), benzophenone, 2,2-dimethoxy-1,2-diphenylethan-1-one, 1-hydroxycyclohexyl phenyl ketone, 2-hydroxy-2-methyl-1-phenylpropan-1-one, 2,4,6-trimethylbenzoyl diphenylphosphine oxide, 1-[4-(2-hydroxyethoxy)-phenyl]-2-hydroxy-2-methyl-1-propan-1-one, and the like, and compounds which generate radicals by light with a wavelength of 360 nm or more such as substances obtained by mixing a thiopyrylium salt-based, merocyanine-based, quinoline-based, or styrylquinoline-based dye with 2,4-diethyl thioxanthone, isopropyl thioxanthone, 1-chloro-4-propoxythioxanthone, 2-(3-dimethylamino-2-hydroxypropoxy)-3,4-dimethyl-9H-thioxanthon-9-one methochloride, 2-methyl-1[4-(methylthio)phenyl]-2-morpholinopropane-1,2-benzyl-2-dimethylamino-1-(4-morpholinophenyl)-butanone-1, bis(cyclopentadienyl)-bis(2,6-difluoro-3-(pyl-1-yl) titanium, or a peroxy ester such as 1,3-di(t-butylperoxycarbonyl)benzene or 3,3′, 4,4′-tetra-(t-butylperoxycarbonyl)benzophenone. Hydrogen peroxide or a persulfate can also be used as a redox-based initiator in combination with, for example, a reducing substance such as a sulfite or L-ascorbic acid, an amine salt, or the like.

As the crosslinking agent, a compound having two or more polymerizable functional groups can be used, and specific examples thereof include ethylene glycol, propylene glycol, trimethylolpropane, glycerin, polyoxyethylene glycol, polyoxypropylene glycol, polyglycerin, N,N′-methylenebisacrylamide, N,N-methylene-bis-N-vinylacetamide, N,N-butylene-bis-N-vinylacetamide, tolylene diisocyanate, hexamethylene diisocyanate, allylated starch, allylated cellulose, diallyl phthalate, tetraallyloxyethane, pentaerythritol triallyl ether, trimethylolpropane triallyl ether, diethylene glycol diallyl ether, and triallyl trimellitate.

The stimulus-responsive gel may contain a plurality of different types of polymer materials.

For example, the stimulus-responsive gel may contain as the polymer material, a first polymer having an OH group (a hydroxy group which bonds to a carbon atom) and a second polymer having a phenylboronic acid structure.

According to this, the stimulus-responsive gel can be in a first state where the OH group possessed by the first polymer and the phenylboronic acid structure possessed by the second polymer are bonded to each other, and in a second state where the bond between the OH group possessed by the first polymer and the phenylboronic acid structure possessed by the second polymer is dissociated.

The change between these states occurs by a change in the surrounding environment (a change in the presence or absence of a given substance (specific component) or a change in the concentration or the like of a given substance (specific component)). Then, the ratio of molecules which are in the first state to molecules which are in the second state among the many molecules (the first polymer molecules and the second polymer molecules) contained in the stimulus-responsive gel changes with inclination according to the strength of the stimulus (the concentration of a given substance (specific component).

Due to this, the strength of the stimulus can be quantitatively detected.

It is preferred that the stimulus-responsive gel is transformed to the second state by reacting the phenylboronic acid structure possessed by the second polymer with lactic acid.

The stimulus-responsive gel containing the first polymer and the second polymer as described above can detect and quantitatively determine lactic acid among various substances (specific components) with particularly high sensitivity in a particularly wide concentration range. In the past, the detection and quantitative determination of lactic acid were mostly performed using an enzyme, and there was no gel material capable of being favorably applied to the detection and quantitative determination of lactic acid. In view of this, by using the stimulus-responsive gel for the detection and quantitative determination of lactic acid, the effect of the invention is more remarkably exhibited.

The reason why the stimulus-responsive gel (the stimulus-responsive gel containing the first polymer and the second polymer) as described above shows high sensitivity to lactic acid is considered to be as follows. That is, in the case where the gel material (culture container) is used for detecting lactic acid, when the concentration of lactic acid is low, the ratio of molecules in the first state where the OH group possessed by the first polymer and the phenylboronic acid structure possessed by the second polymer are bonded to each other is increased. On the other hand, when the concentration of lactic acid is increased, the bond between the OH group possessed by the first polymer and the phenylboronic acid structure possessed by the second polymer is replaced by a bond between lactic acid and the phenylboronic acid structure with extremely high reactivity. This is considered to be because lactic acid is a compound which has an α-hydroxycarboxylic acid structure and has particularly high reactivity with a phenylboronic acid structure, and also the size of the molecule is small, and therefore, in the stimulus-responsive gel, lactic acid can easily come close to the phenylboronic acid structure possessed by the second polymer.

Hereinafter, a case where the stimulus-responsive gel contains the first polymer having an OH group (a hydroxy group which bonds to a carbon atom) and the second polymer having a phenylboronic acid structure, in particular, a case where the stimulus-responsive gel is transformed to the second state by reacting the phenylboronic acid structure possessed by the second polymer with lactic acid and is used for the detection and quantitative determination of lactic acid will be mainly described.

### First Polymer

The first polymer has an OH group (hydroxy group).

The OH group may be an OH group introduced after polymerizing a polymerizable compound (a monomer or the like) as a constituent component of the polymer, but is preferably an OH group possessed by a polymerizable compound as a constituent component of the polymer.

According to this, the adjustment of the percentage or the like of the OH group possessed by the first polymer can be easily and reliably performed.

Examples of the monomer having an OH group which constitutes the first polymer include N-hydroxyethyl acrylamide, N,N′-(1, 2-dihydroxyethylene)bisacrylamide, 2-hydroxyethyl methacrylate, glycerol monomethacrylate, 2-hydroxy-1-acryloxy-3-methacryloxypropane, and 2-hydroxy-1,3-dimethacryloxypropane, and it is possible to use one member or two or more members in combination selected from these, however, the first polymer preferably contains N-hydroxyethyl acrylamide as a constituent component.

According to this, the transformation between the first state and the second state according to the change in the environment (particularly the change in the concentration of lactic acid) in which the stimulus-responsive gel is placed is more favorably performed, and the detection and quantitative determination of the strength of a stimulus (particularly, the concentration of lactic acid) can be more stably performed in a wider range. In addition, the ability to retain the solvent of the stimulus-responsive gel can be made particularly excellent, and thus, a favorable gel state can be stably maintained over a long period of time.

The first polymer may contain a monomer having no OH group as a constituent component thereof. According to this, the percentage or the like of the OH group possessed by the first polymer can be adjusted to a favorable level.

Examples of the monomer having no OH group which constitutes the first polymer include acrylamide, N-methylacrylamide, N-isopropylacrylamide, N,N-dimethylacrylamide, N,N-dimethylaminopropyl acrylamide, various quaternary salts of N,N-dimethylaminopropyl acrylamide, acryloylmorpholine, various quaternary salts of N,N-dimethylaminoethyl acrylate, acrylic acid, various alkyl acrylates, methacrylic acid, various alkyl methacrylates, N-vinylpyrrolidone, acrylonitrile, styrene, polyethyleneglycol diacrylate, 1,6-hexanediol diacrylate, neopentyl glycol diacrylate, tripropylene glycol diacrylate, polypropylene glycol diacrylate, 2,2-bis[4-(acryloxydiethoxy)phenyl]propane, 2,2-bis[4-(acryloxypolyethoxy)phenyl]propane, 2,2-bis[4-(acryloxypolypropoxy)phenyl]propane, ethylene glycol dimethacrylate, diethylene glycol dimethacrylate, triethylene glycol dimethacrylate, polyethylene glycol dimethacrylate, 1,3-butylene glycol dimethacrylate, 1,6-hexanediol dimethacrylate, neopentyl glycol dimethacrylate, polypropylene glycol dimethacrylate, 2,2-bis[4-(methacryloxyethoxy)phenyl]propane, 2,2-bis[4-(methacryloxyethoxydiethoxy)phenyl]propane, 2,2-bis[4-(methacryloxyethoxypolyethoxy)phenyl]propane, trimethylolpropane trimethacrylate, tetramethylolmethane trimethacrylate, trimethylolpropane triacrylate, tetramethylolmethane triacrylate, tetramethylolmethane tetraacrylate, dipentaerythritol hexaacrylate, N,N′-methylene bisacrylamide, N,N′-methylene bismethacrylamide, diethylene glycol diallyl ether, divinylbenzene, ethylenebisacrylamide, N-[3-(dimethylamino)propyl]methacrylamide, diacetone acrylamide, N-t-butylacrylamide, N,N-diethylacrylamide, N-isopropylmethacrylamide, N-(butoxymethyl) acrylamide, N-(isobutoxymethyl)acrylamide, N-phenylacrylamide, 2-acrylamide-2-methylpropanesulfonic acid, 4-acrylamidobenzo-18-crown 6-ether, acryloylaminobenzocrown ether, methacryloylaminobenzocrown ether, and 4-vinylbenzocrown ether, and it is possible to use one member or two or more members in combination selected from these.

The content of the monomer having no OH group in the first polymer is preferably 0.1 mol % or more and 40 mol % or less, more preferably 0.2 mol % or more and 30 mol % or less, further more preferably 0.3 mol % or more and 20 mol % or less.

The first polymer may contain a crosslinking agent component as a constituent component.

According to this, the first polymer has a crosslinked structure, and thus has a three-dimensional network structure. As a result, the ability to retain the solvent of the stimulus-responsive gel can be made particularly excellent, and thus, a favorable gel state can be stably maintained over a long period of time. That is, the stimulus-responsive gel has excellent durability.

As the crosslinking agent component, a compound having two or more polymerizable functional groups can be used, and specific examples thereof include ethylene glycol, propylene glycol, trimethylol propane, glycerin, polyoxyethylene glycol, polyoxypropylene glycol, polyglycerin, N,N′-methylene bisacrylamide, N,N-methylene-bis-N-vinylacetamide, N,N-butylene-bis-N-vinylacetamide, tolylene diisocyanate, hexamethylene diisocyanate, allylated starch, allylated cellulose, diallyl phthalate, tetraallyloxyethane, pentaerythritol triallyl ether, trimethylol propane triallyl ether, diethylene glycol diallyl ether, and triallyl trimellitate, and it is possible to use one member or two or more members in combination selected from these.

The content of the crosslinking agent component in the first polymer is preferably 0.5 mol % or more and 7.0 mol % or less, more preferably 0.8 mol % or more and 6.0 mol % or less, further more preferably 1.1 mol % or more and 5.0 mol % or less.

According to this, the degree of crosslinking of the first polymer can be made to fall within a more favorable range, and while more remarkably exhibiting the effect as described above, the flexibility of the first polymer can be made more suitable.

The content of the monomer having an OH group in the first polymer is preferably 54 mol % or more and 99 mol % or less, more preferably 65 mol % or more and 98.5 mol % or less, further more preferably 76 mol % or more and 98 mol % or less.

According to this, while more remarkably exhibiting the effect of the first polymer because of having an OH group as described above, the effect of the components (the crosslinking agent, the monomer having no OH group, etc.) other than the monomer having an OH group can be sufficiently exhibited.

The hydroxyl value of the first polymer is preferably 15 mgKOH/g or more and 620 mgKOH/g or less, more preferably 34 mgKOH/g or more and 78 mgKOH/g or less.

According to this, while more remarkably exhibiting the effect of the first polymer because of having an OH group as described above, the durability of the stimulus-responsive gel can be made particularly excellent.

On the other hand, when the hydroxyl value of the first polymer is less than the above lower limit, the effect of the first polymer because of having an OH group as described above may not be sufficiently obtained depending on the percentage or the like of the phenylboronic acid structure possessed by the second polymer.

Further, when the hydroxyl value of the first polymer exceeds the above upper limit, the durability of the stimulus-responsive gel is decreased.

The first polymer does not have a phenylboronic acid structure.

The content X₁ of the first polymer in the stimulus-responsive gel is preferably 0.05 mass % or more and 98 mass % or less, more preferably 0.1 mass % or more and 70 mass % or less.

According to this, the sensitivity and quantitativeness with respect to lactic acid can be made particularly excellent, and also the ability to retain the solvent of the stimulus-responsive gel can be made particularly excellent, and thus, a favorable gel state can be stably maintained over a long period of time.

In addition, the content of the first polymer in the polymer material is preferably 1.0 mass % or more and 99 mass % or less, more preferably 1.5 mass % or more and 98 mass % or less.

According to this, particularly excellent sensitivity and quantitativeness with respect to lactic acid are obtained, and also the ability to retain the solvent of the stimulus-responsive gel can be made particularly excellent, and thus, a favorable gel state can be stably maintained over a long period of time.

### Second Polymer

The second polymer has a phenylboronic acid structure.

By including such a second polymer along with the above-mentioned first polymer, the detection of the strength of a stimulus (the concentration or the like of a given component) in a wide range can be easily and stably performed. In particular, in the case where the gel material (culture container) is used for performing the detection and quantitative determination of lactic acid, the sensitivity in a low concentration range (for example, in a range of 0.4 mass % or less) can be made excellent.

The phenylboronic acid structure may be a phenylboronic acid structure introduced after polymerizing a polymerizable compound (a monomer or the like) as a constituent component of the polymer, but is preferably a phenylboronic acid structure possessed by a polymerizable compound as a constituent component of the polymer.

According to this, the adjustment of the percentage or the like of the phenylboronic acid structure possessed by the second polymer can be easily and reliably performed.

Examples of the monomer having a phenylboronic acid structure which constitutes the second polymer include 3-acrylamidophenylboronic acid, vinylphenylboronic acid, acryloyloxyphenylboronic acid, acryloylaminobenzeneboronic acid, methacryloylaminobenzeneboronic acid, and 4-vinylbenzeneboronic acid, and it is possible to use one member or two or more members in combination selected from these, however, the second polymer preferably contains 3-acrylamidophenylboronic acid as a constituent component.

According to this, the transformation between the first state and the second state according to the change in the environment (particularly the change in the concentration of lactic acid) in which the stimulus-responsive gel is placed is more favorably performed, and the detection and quantitative determination of the strength of a stimulus (particularly, the concentration of lactic acid) can be more stably performed in a wider range. In addition, the ability to retain the solvent of the stimulus-responsive gel can be made particularly excellent, and thus, a favorable gel state can be stably maintained over a long period of time.

The second polymer may contain a monomer having no phenylboronic acid structure as a constituent component thereof. According to this, the percentage or the like of the phenylboronic acid structure possessed by the second polymer can be adjusted to a favorable level.

Examples of the monomer having no phenylboronic acid structure which constitutes the second polymer include acrylamide, N-methylacrylamide, N-isopropylacrylamide, N,N-dimethylacrylamide, N,N-dimethylaminopropyl acrylamide, various quaternary salts of N,N-dimethylaminopropyl acrylamide, acryloylmorpholine, various quaternary salts of N,N-dimethylaminoethyl acrylate, acrylic acid, various alkyl acrylates, methacrylic acid, various alkyl methacrylates, N-vinylpyrrolidone, acrylonitrile, styrene, polyethyleneglycol diacrylate, 1,6-hexanediol diacrylate, neopentyl glycol diacrylate, tripropylene glycol diacrylate, polypropylene glycol diacrylate, 2,2-bis[4-(acryloxydiethoxy)phenyl]propane, 2,2-bis[4-(acryloxypolyethoxy)phenyl]propane, 2,2-bis[4-(acryloxypolypropoxy)phenyl]propane, ethylene glycol dimethacrylate, diethylene glycol dimethacrylate, triethylene glycol dimethacrylate, polyethylene glycol dimethacrylate, 1,3-butylene glycol dimethacrylate, 1,6-hexanediol dimethacrylate, neopentyl glycol dimethacrylate, polypropylene glycol dimethacrylate, 2,2-bis[4-(methacryloxyethoxy)phenyl]propane, 2,2-bis[4-(methacryloxyethoxydiethoxy)phenyl]propane, 2,2-bis[4-(methacryloxyethoxypolyethoxy)phenyl]propane, trimethylolpropane trimethacrylate, tetramethylolmethane trimethacrylate, trimethylolpropane triacrylate, tetramethylolmethane triacrylate, tetramethylolmethane tetraacrylate, dipentaerythritol hexaacrylate, N,N′-methylene bisacrylamide, N,N′-methylene bismethacrylamide, diethylene glycol diallyl ether, divinylbenzene, ethylenebisacrylamide, N-[3-(dimethylamino)propyl]methacrylamide, diacetone acrylamide, N-t-butylacrylamide, N,N-diethylacrylamide, N-isopropylmethacrylamide, N-(butoxymethyl)acrylamide, N-(isobutoxymethyl)acrylamide, N-phenylacrylamide, N-hydroxyethylacrylamide, 2-hydroxyethylmethacrylate, glycerol monomethacrylate, 2-hydroxy-1-acryloxy-3-methacryloxypropane, 2-hydroxy-1,3-dimethacryloxypropane, 4-acrylamidobenzo-18-crown 6-ether, acryloylaminobenzocrown ether, methacryloylaminobenzocrown ether, and 4-vinylbenzocrown ether, and it is possible to use one member or two or more members in combination selected from these, however, the second polymer preferably contains N-hydroxyethylacrylamide as a constituent component.

According to this, the affinity between the first polymer and the second polymer can be made more favorable, and a problem of undesirable phase separation or the like in the stimulus-responsive gel can be more reliably prevented over a longer period of time, and the durability and reliability of the stimulus-responsive gel can be made particularly excellent. In addition, the ability to retain the solvent of the stimulus-responsive gel can be made particularly excellent.

The content of the monomer having no phenylboronic acid structure in the second polymer is preferably 1 mol % or more and 96 mol % or less, more preferably 5 mol % or more and 95 mol % or less, further more preferably 20 mol % or more and 93 mol % or less.

The second polymer may contain a crosslinking agent component as a constituent component.

According to this, the second polymer has a crosslinked structure, and thus has a three-dimensional network structure. As a result, the ability to retain the solvent of the stimulus-responsive gel can be made particularly excellent, and thus, a favorable gel state can be stably maintained over a long period of time. That is, the stimulus-responsive gel has excellent durability.

As the crosslinking agent component, a compound having two or more polymerizable functional groups can be used, and specific examples thereof include ethylene glycol, propylene glycol, trimethylol propane, glycerin, polyoxyethylene glycol, polyoxypropylene glycol, polyglycerin, N,N′-methylene bisacrylamide, N,N-methylene-bis-N-vinylacetamide, N,N-butylene-bis-N-vinylacetamide, tolylene diisocyanate, hexamethylene diisocyanate, allylated starch, allylated cellulose, diallyl phthalate, tetraallyloxyethane, pentaerythritol triallyl ether, trimethylol propane triallyl ether, diethylene glycol diallyl ether, and triallyl trimellitate, and it is possible to use one member or two or more members in combination selected from these.

The content of the crosslinking agent component in the second polymer is preferably 0.5 mol % or more and 10.0 mol % or less, more preferably 0.8 mol % or more and 8.0 mol % or less, further more preferably 1.1 mol % or more and 6.0 mol % or less.

According to this, the degree of crosslinking of the second polymer can be made to fall within a more favorable range, and while more remarkably exhibiting the effect as described above, the flexibility of the second polymer can be made more suitable.

The content of the monomer having a phenylboronic acid structure in the second polymer is preferably 3.0 mol % or more and 98 mol % or less, more preferably 3.5 mol % or more and 70 mol % or less, further more preferably 3.8 mol % or more and 70 mol % or less.

According to this, the stimulus-responsive gel has particularly excellent flexibility, and therefore has particularly excellent sensitivity and quantitativeness with respect to a given substance (specific component), and the detection and quantitative determination of the strength of a stimulus (particularly, the concentration of lactic acid) can be more stably performed in a wider range.

On the other hand, when the content of the monomer having a phenylboronic acid structure in the second polymer is less than the above lower limit, it may be difficult to make the range (for example, the concentration of lactic acid) in which the detection and quantitative determination of the strength of a stimulus (for example, the concentration of lactic acid) can be favorably performed sufficiently wide depending on the percentage or the like of the OH group possessed by the first polymer.

In addition, when the content of the monomer having a phenylboronic acid structure in the second polymer exceeds the above upper limit, the stimulus-responsive gel is hard to deform, and therefore, the sensitivity and quantitativeness with respect to a given substance (specific component) are deteriorated.

This is considered to be because the percentage of the phenylboronic acid structure is increased, so that the Π-Π electron interaction (Π-Π stacking) between a plurality of benzene rings strongly appears, and therefore a space into which the solvent or the like enters is reduced.

The content X₂ of the second polymer in the stimulus-responsive gel is preferably 0.01 mass % or more and 70 mass % or less, more preferably 0.05 mass % or more and 65 mass % or less.

According to this, the stimulus-responsive gel has particularly excellent flexibility, and therefore has particularly excellent sensitivity and quantitativeness with respect to a given substance (specific component), and the detection and quantitative determination of the strength of a stimulus (particularly, the concentration of lactic acid) can be more stably performed in a wider range.

On the other hand, when the content X₂ of the second polymer in the stimulus-responsive gel is less than the above lower limit, it may be difficult to make the range (for example, the concentration of lactic acid) in which the detection and quantitative determination of the strength of a stimulus (for example, the concentration of lactic acid) can be favorably performed sufficiently wide depending on the percentage or the like of the OH group possessed by the first polymer.

In addition, when the content X₂ of the second polymer in the stimulus-responsive gel exceeds the above upper limit, the stimulus-responsive gel is hard to deform, and therefore, the sensitivity and quantitativeness with respect to a given substance (specific component) are deteriorated.

Further, the content of the second polymer in the polymer material is preferably 1.0 mass % or more and 70 mass % or less, more preferably 2.0 mass % or more and 65 mass % or less.

According to this, the stimulus-responsive gel has particularly excellent flexibility, and therefore has particularly excellent sensitivity and quantitativeness with respect to a given substance (specific component), and the detection and quantitative determination of the strength of a stimulus (particularly, the concentration of lactic acid) can be more stably performed in a wider range.

On the other hand, when the content of the second polymer in the polymer material is less than the above lower limit, it may be difficult to make the range (for example, the concentration of lactic acid) in which the detection and quantitative determination of the strength of a stimulus (for example, the concentration of lactic acid) can be favorably performed sufficiently wide depending on the percentage or the like of the OH group possessed by the first polymer.

In addition, when the content of the second polymer in the polymer material exceeds the above upper limit, the stimulus-responsive gel is hard to deform, and therefore, the sensitivity and quantitativeness with respect to a given substance (specific component) are deteriorated.

When the content of the first polymer in the stimulus-responsive gel is represented by X₁ (mass %) and the content of the second polymer in the stimulus-responsive gel is represented by X₂ (mass %), X₁ and X₂ preferably satisfy the following relationship: 0.2≦X₂/X₁≦8, more preferably satisfy the following relationship: 1.3≦X₂/X₁≦1.9.

According to this, the stimulus-responsive gel has particularly excellent flexibility, and therefore has particularly excellent sensitivity and quantitativeness with respect to a given substance (specific component), and the detection and quantitative determination of the strength of a stimulus (particularly, the concentration of lactic acid) can be more stably performed in a wider range.

On the other hand, when the value of X₂/X₁ is less than the above lower limit, it may be difficult to make the range (for example, the concentration of lactic acid) in which the detection and quantitative determination of the strength of a stimulus (for example, the concentration of lactic acid) can be favorably performed sufficiently wide.

In addition, when the value of X₂/X₁ exceeds the above upper limit, the stimulus-responsive gel is hard to deform, and therefore, the sensitivity and quantitativeness with respect to a given substance (specific component) are deteriorated.

The content of the polymer material in the stimulus-responsive gel is preferably 0.7 mass % or more and 36.0 mass % or less, more preferably 2.4 mass % or more and 27.0 mass % or less.

### Solvent

By including the solvent in the stimulus-responsive gel, the above-mentioned polymer material can be favorably gelled.

As the solvent, any of various types of organic solvents and inorganic solvents can be used. Specific examples thereof include water; various types of alcohols such as methanol and ethanol; ketones such as acetone; ethers such as tetrahydrofuran and diethyl ether; amides such as dimethylformamide; chain aliphatic hydrocarbons such as n-pentane, n-hexane, n-heptane, and n-octane; alicyclic hydrocarbons such as cyclohexane and methylcyclohexane; and aromatic hydrocarbons such as benzene, toluene, and xylene, however, in particular, a solvent containing water is preferred.

The stimulus-responsive gel may contain a plurality of different types of components as the solvent.

The content of the solvent in the stimulus-responsive gel is preferably 30 mass % or more and 95 mass % or less, more preferably 50 mass % or more and 90 mass % or less.

### Particles

The gel material constituting the gel section 2 may contain a component other than the above-mentioned components (another component).

The gel material constituting the gel section 2 may contain particles (fine particles) having an average particle diameter of 10 nm or more and 1000 nm or less.

According to this, the structural color caused by Bragg reflection can be visually recognized. In particular, a distance between adjacent particles changes accompanying the expansion or contraction of the stimulus-responsive gel, and the structural color caused by Bragg reflection also changes, and therefore, a given substance (specific component) can be detected optically (visually). In addition, since the structural color caused by Bragg reflection or the change in the structural color is visually recognized, for example, by the color tone thereof, also the quantitative determination of a given substance (specific component) can be more easily and also more accurately performed. Further, even without using a detection device for detecting the deformation (expansion or contraction) of the stimulus-responsive gel, the detection and quantitative determination of a given substance (specific component) can be favorably performed by visual observation. Further, even in the case of using a detection device, it is not necessary to detect a minute change in the shape, but is only necessary to detect a change in the color tone, and therefore, as the detection device, a relatively inexpensive device can be used.

In the case where the gel material constituting the gel section 2 contains particles, the average particle diameter of the particles is preferably 10 nm or more and 1000 nm or less, and more preferably 20 nm or more and 500 nm or less.

The “average particle diameter” as used herein refers to an average particle diameter on the volume basis, and can be obtained by, for example, performing measurement using a particle size distribution analyzer employing a Coulter counter method (model: TA-II, manufactured by Coulter Electronics, Inc.) with an aperture of 50 μm for a dispersion liquid obtained by adding a sample to methanol and dispersing the sample therein for 3 minutes with an ultrasonic disperser.

Examples of the constituent material of the fine particles include inorganic materials such as silica and titanium oxide; and organic materials (polymers) such as polystyrene, polyester, polyimide, polyolefin, poly(methyl (meth)acrylate), polyethylene, polypropylene, polyether sulfone, nylon, polyurethane, polyvinyl chloride, and polyvinylidene chloride, however, the fine particles are preferably silica fine particles.

According to this, the shape stability and the like of the fine particles are made particularly excellent, and the durability, reliability, and the like of the gel material and the culture container can be made particularly excellent. In addition, the silica fine particles are relatively easily available as those having a sharp particle size distribution (monodispersed fine particles), and therefore are advantageous also from the viewpoint of stable production and supply of the gel material and the culture container.

The shape of the fine particle is not particularly limited, but is preferably a spherical shape. According to this, the structural color caused by colloidal crystals can be more reliably visually recognized, and therefore, the quantitative determination of the concentration of lactic acid can be more easily and also more reliably performed.

The gel material may contain a plurality of different types of fine particles.

The content of the fine particles in the gel material is preferably 1.6 mass % or more and 36 mass % or less, more preferably 4.0 mass % or more and 24 mass % or less.

According to this, the effect of including the fine particles in the gel material as described above is more remarkably exhibited.

### Another Component

The gel material constituting the gel section 2 may contain a component other than the above-mentioned components (another component).

Examples of such a component include a coloring agent, a slipping agent (leveling agent), an antifungal agent, a preservative, an antioxidant, a solvent which does not form a hydrogen bond, and a moisturizing agent.

### Culture System

Hereinafter, a culture system will be described.

FIG. 2 is a schematic perspective view for illustrating a preferred embodiment of a culture system.

As shown in FIG. 2, a culture system 100 includes a culture container 10 and a detection unit 20 that detects the state of a gel section 2 of the culture container 10.

According to this, while preventing contamination of the inside of the culture container, the deformation (expansion or contraction) of the stimulus-responsive gel in response to a stimulus by a given substance can be stably detected, and thus, monitoring of the inside of the culture container can be favorably performed.

As the detection unit 20, for example, a light detector (an optical sensor or the like) that detects the color tone (the wavelength of reflected light) of the gel section 2 constituted by a material containing a stimulus-responsive gel or the like can be used.

According to this, for example, in the case where the gel material constituting the gel section 2 contains particles as described above, the structural color caused by Bragg reflection or the change in the structural color can be favorably detected, and the stimulus by a given substance (the concentration or the like of a specific substance) can be detected with higher accuracy, and also the detection rate can be increased.

In the configuration shown in the drawing, the detection unit 20 is disposed on the bottom surface side of the culture container 10 (container main body 1).

According to this, for example, the color tone of the culture solution or the like can be effectively prevented from adversely affecting the detection of a given substance. Further, by disposing the culture container 10 on the detection unit 20, the size of the culture system 100 (culture device) can be reduced. In addition, the workability when the culture container 10 is provided in a given place in the culture system 100 (culture device) can be made excellent.

The culture system 100 may also include a light source (not shown).

Accordingly, the detection of reflected light can be stably performed, and the reliability of the detection result can be made more excellent.

As the light source, any light source may be used, however, in the case where the gel section 2 exhibits structural color caused by Bragg reflection, a light source which irradiates light in a wavelength range including the peak wavelength of reflected light when the stimulus-responsive gel is in an expanded state and the peak wavelength of reflected light when the stimulus-responsive gel is in a contracted state is preferred.

The light source may be integrally provided with the detection unit 20 (unitized with the detection unit 20).

Further, a configuration in which the detection result detected by the detection unit 20 is sent to a control section (not shown) by wire or wireless may be adopted, and the control section may be a control section that controls the culture conditions (for example, the temperature, the concentration of oxygen, etc.) based on the detection result.

Further, the detection result may be recorded in a recording section (not shown). According to this, for example, the change over time in the state of the gel section 2 can be examined in detail. In addition, the recorded detection result can be utilized for setting the conditions of the culture to be performed thereafter, and thus, the culture efficiency can be further improved.

The detection unit 20 may be incorporated in a robot, and the robot may perform detection of the state of the gel section 2.

According to this, the culture process can be performed in an unattended manner, and thus, the contamination of the inside of the culture container can be more effectively prevented, and also the culture efficiency can be further improved. Further, culture medium replacement, observation of cells with a microscope, or the like can also be performed by a robot, and the detection by the detection unit 20 can be a part of an automated cell culture process performed by a robot, and thus, the culture efficiency can be made particularly excellent.

Further, the culture system 100 may be, for example, a system which performs culture using a plurality of culture containers 10, and may be configured to automatically perform introduction of a culture solution, cells, and the like into the plurality of culture containers 10, detection of the state of the gel section 2 by the detection unit 20, and the like.

According to this, the culture efficiency can be further improved.

Further, the plurality of culture containers 10 may be conveyed by a conveyance unit such as a belt conveyer, and the detection of the state of the gel section 2 by the detection unit 20 may be performed during the conveyance process.

Hereinabove, preferred embodiments have been described, however, the invention is not limited thereto.

For example, the culture container may have a configuration other than those described above.

Further, in the above-mentioned embodiments, a case where the culture container has a dish shape (particularly, a petri dish shape) has been mainly described, however, the shape of the culture container is not limited thereto, and may be any shape, for example, a bag shape, a fiber shape, or the like.

Further, in the above-mentioned embodiments, a case where the gel section has a sheet shape has been representatively described, however, the gel section may have any shape other than the sheet shape.

Further, in the above-mentioned embodiments, a case where the gel section is provided on an inner bottom surface of the container main body has been representatively described, however, the place where the gel section is provided is not limited thereto.

In addition, in the culture container, some constituent members may be configured to be replaceable or may be configured to be detachable and reattachable. For example, the gel section may be configured to be replaceable or detachable and reattachable.

Further, in the above-mentioned embodiments, a case where the culture container includes a culture container main body and a gel section has been representatively described, however, the entirety of the culture container may be constituted by a gel section (gel material).

The entire disclosure of Japanese Patent Application No. 2015-107155 filed May 27, 2015 is expressly incorporated by reference herein.

What is claimed is:

1. A culture container, comprising a gel section constituted by a material containing a stimulus-responsive gel which expands or contracts in response to a stimulus by a given substance in at least a portion of a part coming into contact with a culture solution.

2. The culture container according to claim 1, wherein
the culture container includes a container main body and the gel section, and
the gel section is provided on an inner bottom surface of the container main body.

3. The culture container according to claim 1, wherein
the culture container includes a container main body and the gel section, and
at least the part of the container main body where the gel section is provided is constituted by a material having light transmittance.

4. The culture container according to claim 1, wherein the stimulus-responsive gel includes a first polymer having an OH group and a second polymer having a phenylboronic acid structure as polymer materials.

5. The culture container according to claim 4, wherein the stimulus-responsive gel is transformed to a second state where the bond between the OH group possessed by the first polymer and the phenylboronic acid structure possessed by the second polymer is dissociated by reacting the phenylboronic acid structure possessed by the second polymer with lactic acid.

6. The culture container according to claim 4, wherein the first polymer contains N-hydroxyethylacrylamide as a constituent component.

7. The culture container according to claim 4, wherein the second polymer contains a monomer having a phenylboronic acid structure as a constituent component.

8. The culture container according to claim 4, wherein the second polymer contains 3-acrylamidophenylboronic acid as a constituent component.

9. The culture container according to claim 4, wherein when the content of the first polymer is represented by X₁ (mass %) and the content of the second polymer is represented by X₂ (mass %), the following relationship is satisfied: 0.2≦X₂/X₁≦8.

10. The culture container according to claim 1, wherein the gel section contains the stimulus-responsive gel and fine particles having an average particle diameter of 10 nm or more and 1000 nm or less.

11. A gel material, which is a material constituting a region containing at least a portion of a part coming into contact with a culture solution of a culture container, comprising a stimulus-responsive gel which expands or contracts in response to a stimulus by a given substance.

12. A culture system, comprising:
the culture container according to claim 1; and
a detection unit that detects the state of the gel section.

13. A culture system, comprising:
the culture container according to claim 2; and
a detection unit that detects the state of the gel section.

14. A culture system, comprising:
the culture container according to claim 3; and
a detection unit that detects the state of the gel section.

15. A culture system, comprising:
the culture container according to claim 4; and
a detection unit that detects the state of the gel section.

16. A culture system, comprising:
the culture container according to claim 5; and
a detection unit that detects the state of the gel section.

17. A culture system, comprising:
the culture container according to claim 6; and
a detection unit that detects the state of the gel section.

18. A culture system, comprising:
the culture container according to claim 7; and
a detection unit that detects the state of the gel section.

19. A culture system, comprising:
the culture container according to claim 8; and
a detection unit that detects the state of the gel section.

20. A culture system, comprising:
the culture container according to claim 9; and
a detection unit that detects the state of the gel section.
